(19) United States
(12) Patent Application Publication
(10) Pub. No.: US 2026/0109710 A1
(43) Pub. Date: Apr. 23, 2026

(54) SYNTHESIS OF BROMODOMAIN INHIBITORS

(71) Applicant: Constellation Pharmaceuticals, Inc., Boston, MA (US)

(72) Inventors: Louis Plamondon, Cambridge, MA (US); Jatin Patel, Boston, MA (US); Yi Wang, Boston, MA (US); Cuixiang Sun, Arlington, MA (US)

(21) Appl. No.: 18/987,624

(22) Filed: Dec. 19, 2024

Related U.S. Application Data
(60) Provisional application No. 63/612,455, filed on Dec. 20, 2023.

(51) Int. Cl. C07D 498/04; C07D 261/08; C07D 261/10

(52) U.S. Cl. CPC C07D 498/04; C07D 261/08; C07D 261/10

(57) ABSTRACT

Provided herein are processes which are useful for the preparation of small molecule bromodomain inhibitors.

### RELATED APPLICATIONS

This application claims the benefit of priority to U.S. Provisional Application No. 63/612,455, filed Dec. 20, 2023, the entire contents of which are incorporated herein by reference.

### BACKGROUND

The bromodomain (BRD) family of proteins are involved in a diverse range of functions and regulate gene expression thorough histone acetylation, histone recognition, chromatin remodeling, and transcriptional machinery regulation. BRD-containing proteins are often deregulated in cancer and small molecule inhibitors that target the bromodomain and extraterminal domain (BET) family of BRDs are currently in clinical trials. Recently, the BET family of proteins, along with the JAK/STAT pathway, have been implicated in the development and progression of myelofibrosis. Both pathways may increase levels of pro-inflammatory cytokines and stimulate production of abnormal blood cell precursors called megakaryocytes. The BET family of proteins is emerging as a promising therapeutic target whose modulation may alter the underlying cause of disease in myelofibrosis, with possible synergistic results alongside JAK inhibition.

Compounds which are designed to inhibit BET proteins have been previously described (See e.g., PCT/US2011/063046 and PCT/US2015/036347) and are believed to be useful for treating various cancers such a myelofibrosis as well as for treating certain inflammatory conditions and viral infections. To facilitate the development of new compounds, and to expand the production methods for current BET inhibitors, alternative methods of synthetic preparation are needed.

### SUMMARY

Provided herein are methods of preparing intermediates useful in the preparation of BET inhibitors. In one aspect, such methods comprise a process for preparing a compound having the Formula I:

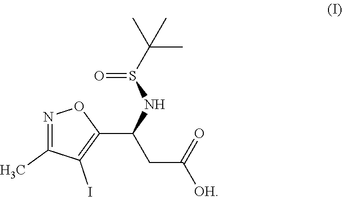 [SMILES: Cc1noc([C@H](CC(=O)O)N[S@@](=O)C(C)(C)C)c1I]

In another aspect, such methods comprise a process for preparing a compound having the Formula II:

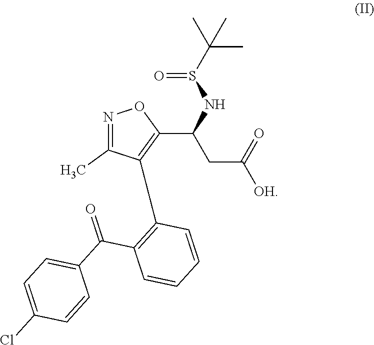 [SMILES: Cc1noc([C@H](CC(=O)O)N[S@@](=O)C(C)(C)C)c1-c1ccccc1C(=O)c1ccc(Cl)cc1]

In another aspect, such methods comprise a process for preparing a compound having the Formula III:

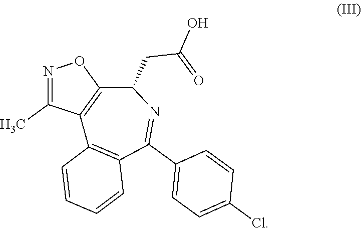 [SMILES: Cc1noc2c1-c1ccccc1C(c1ccc(Cl)cc1)=N[C@H]2CC(=O)O]

Other compounds and methods of preparing such are further described herein.

### DETAILED DESCRIPTION

In embodiments, provided herein is a process for preparing a compound having the Formula I:

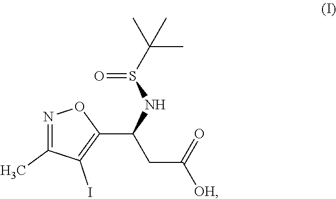 [SMILES: Cc1noc([C@H](CC(=O)O)N[S@@](=O)C(C)(C)C)c1I]

said method comprising reacting a compound having the Formula A:

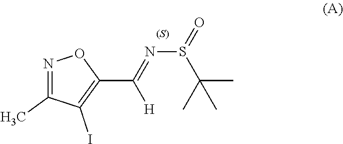

with a strong base in the presence of ethyl acetate followed by ester hydrolysis to form a compound having the Formula I.

In embodiments, for the method of preparing a compound having the Formula I, the strong base is a metal containing base. In embodiments, for the method of preparing a compound having the Formula I, the strong base is a sterically hindered metal containing base. In embodiments, for the method of preparing a compound having the Formula I, the strong base is selected from lithium bis(trimethylsilyl)amide (LiHMDS), sodium bis(trimethylsilyl)amide (NaHMDS), potassium bis(trimethylsilyl)amide (KHMDS), and the like. In embodiments, for the method of preparing a compound having the Formula I, the strong base is potassium bis(trimethylsilyl)amide.

In embodiments, for the method of preparing a compound having the Formula I, the method further comprising reacting the compound having the Formula A with a strong base in the presence of ethyl acetate and at least one organic solvent. In embodiments, for the method of preparing a compound having the Formula I, the method further comprising reacting the compound having the Formula A with a strong base in the presence of ethyl acetate and tetrahydrofuran (THF).

In embodiments, for the method of preparing a compound having the Formula I, said ester hydrolysis is performed under basic conditions. In embodiments, for the method of preparing a compound having the Formula I, said ester hydrolysis is performed using an inorganic hydroxide base. In embodiments, for the method of preparing a compound having the Formula I, said ester hydrolysis is performed using an inorganic hydroxide base selected from lithium hydroxide, sodium hydroxide, potassium hydroxide, and the like. In embodiments, for the method of preparing a compound having the Formula I, said ester hydrolysis is performed using lithium hydroxide. In embodiments, for the method of preparing a compound having the Formula I, said ester hydrolysis is performed under aqueous conditions. In embodiments, for the method of preparing a compound having the Formula I, said ester hydrolysis is performed using lithium hydroxide, water, and ethanol.

In embodiments, provided herein is a process for preparing a compound having the Formula II:

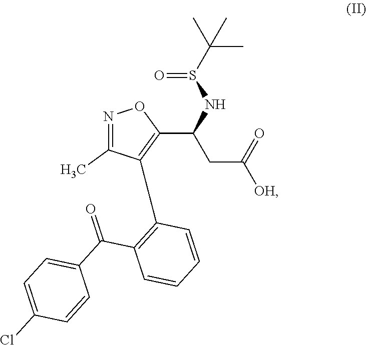 [SMILES: Cc1noc([C@H](CC(=O)O)N[S@@](=O)C(C)(C)C)c1-c1ccccc1C(=O)c1ccc(Cl)cc1]

said method comprising reacting a compound having the Formula I:

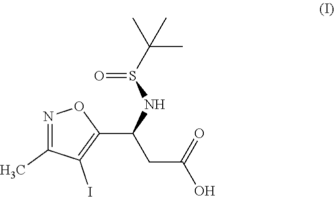 [SMILES: Cc1noc([C@H](CC(=O)O)N[S@@](=O)C(C)(C)C)c1I]

with a compound having the Formula B:

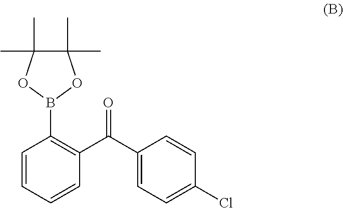 [SMILES: CC1(C)OB(c2ccccc2C(=O)c2ccc(Cl)cc2)OC1(C)C]

in the presence of a palladium source and a base.

In embodiments, for the method of preparing a compound having the Formula II, the palladium source is selected from Pd(Ph₃P)₄, Pd(OAc)₂, Pd₂(dba)₃, Pd(Ph₃)₂Cl₂, Pd(dppf)Cl₂, and the like. In embodiments, for the method of preparing a compound having the Formula II, the palladium source may be combined with one or more suitable phosphine ligands. Such ligands include, e.g., Ph₃P, (t-Bu)₃P, XPhos, SPhos, DPPE, DPPF, BINAP, DPPB, and the like. In embodiments, for the method of preparing a compound having the Formula II, the palladium source is Pd(dppf)Cl₂.

In embodiments, for the method of preparing a compound having the Formula II, the base is an inorganic base. In embodiments, for the method of preparing a compound having the Formula II, the base is selected from Na₂CO₃, K₂CO₃, Cs₂CO₃, t-BuOK, NaOEt, K₃PO₄, and the like. In embodiments, for the method of preparing a compound having the Formula II, the base is K₃PO₄.

In embodiments, for the method of preparing a compound having the Formula II, the reaction of the compound having the Formula I with the compound having the Formula B further comprises the addition of at least one organic solvent. In embodiments, for the method of preparing a compound having the Formula II, the reaction of the compound having the Formula I with the compound having the Formula B further comprises the addition of at least one aprotic organic solvent, selected from THF, 2MeTHF, MTBE, 1,4-dioxane, DME, and the like. In embodiments, for the method of preparing a compound having the Formula II, the reaction of the compound having the Formula I with the compound having the Formula B further comprises the addition of 2MeTHF.

In embodiments, for the method of preparing a compound having the Formula III, the reaction of the compound having the Formula I with the compound having the Formula B further comprises reacting the compound having the Formula II with acid and reacting the resulting intermediate with base to form a compound having the Formula III:

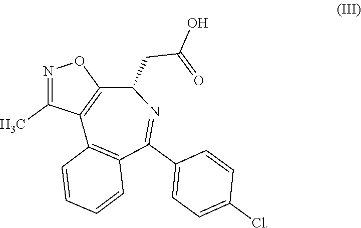 [SMILES: Cc1noc2c1-c1ccccc1C(c1ccc(Cl)cc1)=N[C@H]2CC(=O)O]

In embodiments, for the method of preparing a compound having the Formula III, the acid is an aqueous acid. In embodiments, for the method of preparing a compound having the Formula III, the acid is selected from HCl, HBr, HI, H₃PO₄, and H₂SO₄. In embodiments, for the method of preparing a compound having the Formula III, the acid is HCl. In embodiments, for the method of preparing a compound having the Formula III, the base is an aqueous base. In embodiments, for the method of preparing a compound having the Formula III, the base is selected from Na₂CO₃, K₂CO₃, and K₃PO₄. In embodiments, for the method of preparing a compound having the Formula III, the base is K₃PO₄.

In embodiments, also provided herein is a compound having the Formula I:

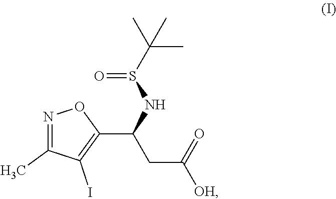 [SMILES: Cc1noc([C@H](CC(=O)O)N[S@@](=O)C(C)(C)C)c1I]

or a salt thereof.

In embodiments, also provided herein is a compound having the Formula II:

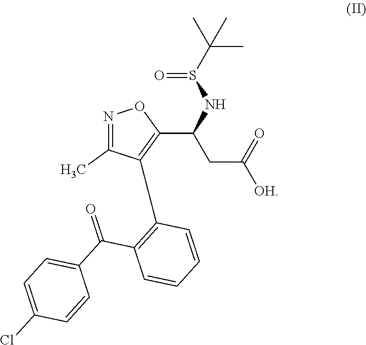 [SMILES: Cc1noc([C@H](CC(=O)O)N[S@@](=O)C(C)(C)C)c1-c1ccccc1C(=O)c1ccc(Cl)cc1]

or a salt thereof.

In embodiments, also provided herein is a compound having the Formula III:

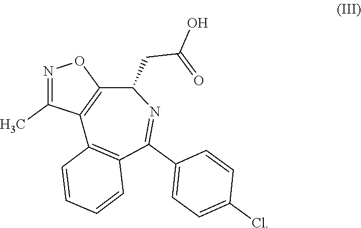 [SMILES: Cc1noc2c1-c1ccccc1C(c1ccc(Cl)cc1)=N[C@H]2CC(=O)O]

or a salt thereof.

Salts are known in the chemical art and include those derived from suitable inorganic and organic acids and bases. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts of the described compounds (including those in the examples) are included as part of the invention.

### EXEMPLIFICATION

### Abbreviations

APhos (4-(N,N-Dimethylamino)phenyl)di-tert-butyl phosphine, A-taPhos, [4-(Dimethylamino)phenyl]bis(tert-butyl)phosphine
aq aqueous
Boc₂O di-tert-butyl dicarbonate
CuSO₄ copper (II) sulfate
EtOAc ethyl acetate
EtOH ethyl alcohol
HCl hydrochloric acid
KHMDS potassium bis(trimethylsilyl)amide
K₃PO₄:3H₂O potassium phosphate trihydrate
LiOH lithium hydroxide
MeTHF 2-methyl tetrahydrofuran
(NH₄)₂CO₃ ammonium carbonate
PinBH pinacolborane
Pd₂(dba)₃ tris(dibenzylideneacetone)dipalladium(0)
Pd(dppf)Cl₂-DCM [1,1′-Bis(diphenylphosphino)ferrocene]dichloropalladium(II)-dichloromethane complex
THF tetrahydrofuran

Procedures for synthesizing intermediates and bromodomain inhibitors are provided below.

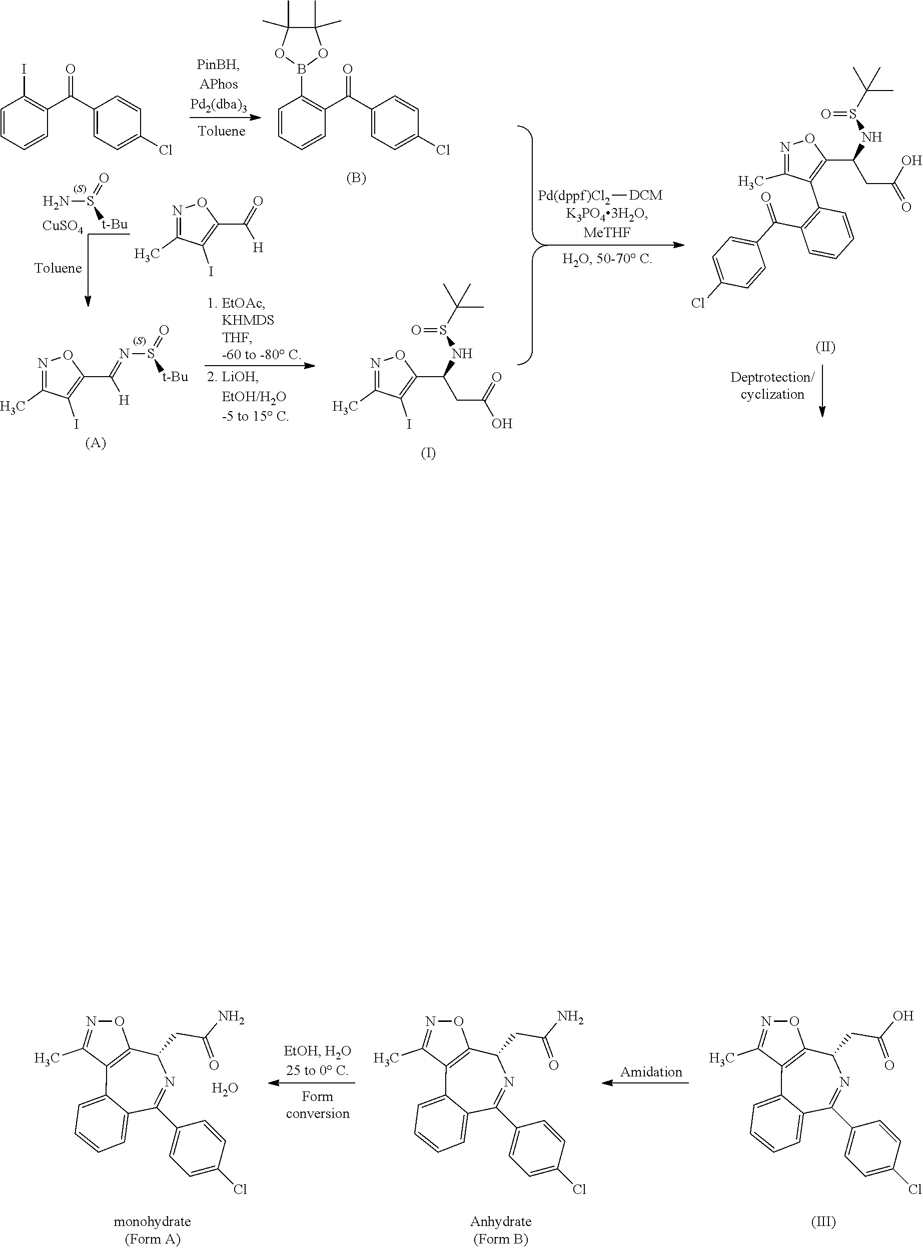

### Preparation of (4-chlorophenyl) (2-(4,4,5,5-tetramethyl-1,3,2-dioxaborolan-2-yl)phenyl) methanone (B)

(4-chlorophenyl) (2-iodophenyl) methanone was dissolved in toluene. To this solution was added triethylamine, pinacolborane, bis(di-tert-butyl)-4-dimethylaminophenylphosphine (APhos) and tris(dibenzylideneacetone)dipalladium (Pd₂(dba)₃) sequentially. The resulting mixture was inert with N₂ and heated to 45-65° C. until reaction was complete by HPLC analysis. The mixture was cooled, quenched with ethanol and filtered. The filter cake was washed with toluene. The combined filtrate was swapped into isopropyl alcohol. The product was crystallized from isopropyl alcohol/n-heptane (approximately 3:1 v/v) at −10 to 10° C. The solid was filtered, washed with isopropyl alcohol/n-heptane (approximately 3:1 v/v), and dried under vacuum at 40 to 60° C. to produce the isolated product B. Typical yield ranged from 84-91 mole percent.

### Preparation of (S,E)-N-((4-iodo-3-methylisoxazol-5-yl)methylene)-2-methylpropane-2-sulfinamide (A)

4-iodo-3-methylisoxazole-5-carbaldehyde was dissolved in toluene. To this solution was added anhydrous copper (II) sulfate, followed by(S)-tert-butanesulfinamide, and acetic acid. The resulting mixture was heated to 43-58° C. until reaction was complete as indicated by HPLC analysis. The mixture was cooled and filtered. The filter cake was washed with MTBE. The combined filtrate was adjusted with NaHCO; to pH 3.5-5.5, washed with water and partitioned. The organic layer was concentrated and crystallized from toluene/MTBE/n-heptane at −10 to 7° C. The solid was filtered, washed with cold toluene/MTBE/n-heptane. The batch was dried at 30 to 55° C. under vacuum to produce the product. Typical yield ranges from 75-84 mole percent.

### Preparation of (S)-3-(((S)-tert-butylsulfinyl)amino)-3-(4-iodo-3-methylisoxazol-5-yl)propanoic acid (I)

Compound A was dissolved in THF at −15-15° C. to form feed solution A. KHMDS (1M in THF) was diluted with THE at −15 to 15° C. to form feed solution B. EtOAc was mixed with THE at ambient temperature to form feed solution C. AcOH was mixed with H₂O at ambient temperature to form quench solution D. After cooling the flow reactors to −60 to −80° C. KHMDS solution (B) and EtOAc solution (C) were charged to the 1st flow reactor to form the ester enolate intermediate. This was followed by the addition of the feed solution (A) in the 2nd flow reactor to form the ethyl ester. The reaction mixture was quenched with AcOH/H₂O (solution D) in continuous stirred tank reactor (CSTR) at 5-30° C. After steady state was reached, the product rich solution was checked for reaction completion. The quenched reaction mixture was collected in multiple containers, if passing quality control, was combined. After phase separation, the organic phase was solvent exchanged into EtOH.

To the abovementioned ethyl ester solution in EtOH, was charged LiOH solution at −5 to 15° C. The reaction was stirred at that temperature until the hydrolysis was complete as determined by HPLC analysis. The reaction was quenched with HCOOH aqueous solution to pH 2.0-5.0, to precipitate the product. The slurry was filtered, and the filter cake was washed with water. The solid was reslurried in MTBE and water at 30-60° C. for at least 0.5 h, then cooled to 0-20° C. After stirring for at least an additional 0.5 h, the product was filtered and washed with cold water and MTBE. The product was dried under vacuum below 65° C. Typical yield ranges from 78-84 mole percent.

### Preparation of (S)-3-(((S)-tert-butylsulfinyl)amino)-3-(4-(2-(4-chlorobenzoyl)phenyl)-3-methylisoxazol-5-yl)propanoic acid (II)

To a solution of K₃PO₄·3H₂O in water, was charged with Compound I, 2-MeTHF and Compound B. After bubbling with N₂, Pd(dppf)Cl₂·DCM was charged. The reaction mixture was sparged with N₂, then stirred at 50-70° C. until reaction completion as indicated by HPLC analysis. Note: The proven acceptable range for Compound B charge is 0.97 to 1.02 eq and needs to be carefully controlled. Excess Compound B charge resulted in the generation of an acid dimer impurity shown below.

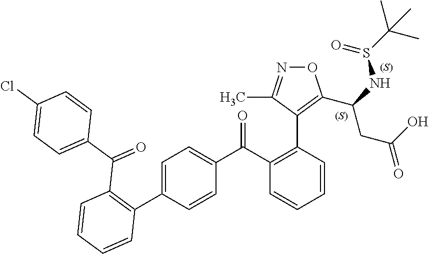

The batch was cooled to room temperature and partitioned. The bottom aqueous layer was discarded, and the product-rich top organic layer was extracted with water. The resulting product-rich aqueous layer was washed with 2-MeTHF and MTBE, followed by the treatment with L-cysteine and 1M NaHSO₃ aqueous solution. The pH was adjusted to 5.7-6.2 to precipitate the product. The 2-MeTHF was added to extract the product and phases were separated. The product-rich organic solution was washed with diluted aqueous NaCl solution. The organic solution was heated to 30 to 50° C., and then treated with activated carbon. It was concentrated under vacuum to approximately 3.5-5.5 volumes below 50° C. The batch was heated to 30 to 50° C. and charged with toluene and n-heptane, then cooled to approximately 10-30° C. to crystallize the product. The resulting slurry was filtered, the filter cake was washed with n-heptane. The wet cake was dried (under vacuum to afford the product. Typical yield ranges from 74-81 mole percent.

### Preparation of (S)-2-(6-(4-chlorophenyl)-1-methyl-4H-benzo[c]isoxazolo[4,5-e]azepin-4-yl)acetic acid (III)

### Deprotection/Cyclization

Compound II was dissolved in 1,4-dioxane and water. To this solution was added concentrated HCl and the resulting mixture is stirred at 15 to 35° C. until deprotection was complete as indicated by HPLC analysis. Toluene was charged, followed by the charge of K₃PO₄ aqueous solution to adjust pH to 1 to 2.5. The reaction was stirred for an additional at least 0.5 h. A second portion of K₃PO₄ aqueous solution was charged to adjust pH to 5 to 6.8. The batch was stirred at 15-35° C. until cyclization was complete. The phases were separated, and the aqueous phase was extracted with toluene. The combined toluene phases were extracted with aqueous K₂CO₃ solution. The product rich basic aqueous solution was treated with activated charcoal at ambient temperature to remove dppf oxide and residual palladium. The resulting aqueous solution was washed with toluene/2-MeTHF mixed solvent followed by heptane wash. The batch was acidified with aqueous acetic acid at room temperature to precipitate out the product. The resulting suspension was stirred at room temperature to produce the free acid product as a solid. The solid was filtered, washed with water and n-heptane, respectively.

### Optional Rework Procedure:

The wet cake was dissolved in 2-MeTHF at 15-35° C. Toluene was added, and the resulting mixture was concentrated under vacuum to 2-3.5 volumes below: 55° C. Additional toluene was added, and the solvent swap process was repeated until the residual 2-MeTHF meets the spec. The resulting mixture was heated at 50-70° C. for not less than 0.5 h and cooled to 10-30° C. to complete the crystallization. The solid was filtered and washed with toluene.

### Drying

The wet cake was dried under vacuum below 60° C. to produce the product. Typical yield ranges from 82-91 mole percent (without rework). The yield for the rework process is about 80 mole percent.

### Preparation of (S)-2-(6-(4-chlorophenyl)-1-methyl-4H-benzo[c]isoxazolo[4,5-e]azepin-4-yl)acetamide (Form B)

### Amidation Reaction:

To a mixture of the product from above, ammonium carbonate, pyridine in 2-MeTHF, was charged with di-tert-butyl pyrocarbonate (Boc anhydride, Boc₂O). The batch was stirred at 10-30° C. until reaction completion. The mixture was washed with diluted aqueous NaCl solution, and the phases are separated. The rich organic layer is treated with activated charcoal at 50-65° C. The product-rich organic solution was concentrated below 65° C. Additional 2-MeTHF was added and concentrated again at below 65° C. This step was repeated until the batch is dry as indicated by KF analysis. The dried 2-MeTHE solution was heated to 55-65° C. and charged with n-heptane. After seeding, additional n-heptane was charged, and the batch is gradually cooled to room temperature. The resulting slurry was stirred at room temperature until crystallization is complete. The product was filtered and washed with n-heptane and dried below 45° C. to give anhydrous Form B. Typical yield ranges from 88-94 mole percent.

### (S)-2-(6-(4-chlorophenyl)-1-methyl-4H-benzo[c]isoxazolo[4,5-e]azepin-4-yl)acetamide monohydrate (Form A)

The anhydrate from above was dissolved in a solution of ethanol and water (approximately 4:1, v/v) at room temperature. The resulting clear solution was polish filtered. Water was added to the filtrate followed by seeding and cooling to −10 to 10° C. to crystallize the product in the desired Form A. The resulting solids were filtered, washed with purified water. The wet cake was dried under humidity to afford the monohydrate Form A product. See e.g., U.S. Pat. No. 9,969,747. The product was micronized via jet milling with nitrogen gas.

While have described a number of embodiments of this, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A process for preparing a compound having the Formula I: 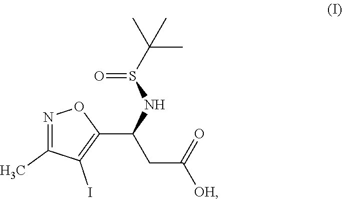 [SMILES: Cc1noc([C@H](CC(=O)O)N[S@@](=O)C(C)(C)C)c1I]
said method comprising reacting a compound having the Formula A: 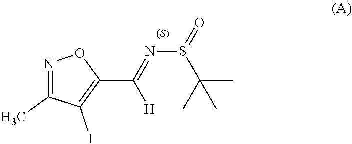
with a strong base in the presence of ethyl acetate followed by ester hydrolysis to form a compound having the Formula I.

2. The process of claim 1, wherein the strong base is a metal containing base.

3. The process of claim 1 or 2, wherein the strong base is a sterically hindered metal containing base.

4. The process of any one of claims 1 to 3, wherein the strong base is selected from lithium bis(trimethylsilyl)amide (LiHMDS) and sodium bis(trimethylsilyl)amide (NaHMDS), and potassium bis(trimethylsilyl)amide (KHMDS).

5. The process of any one of claims 1 to 4, wherein the strong base is potassium bis(trimethylsilyl)amide (KHMDS).

6. The process of any one of claims 1 to 5, further comprising reacting the compound having the Formula A with a strong base in the presence of ethyl acetate and at least one organic solvent.

7. The process of claim 6, wherein the at least one additional solvent is tetrahydrofuran (THF).

8. The process of any one of claims 1 to 7, wherein said ester hydrolysis is performed under basic conditions.

9. The process of any one of claims 1 to 8, wherein said ester hydrolysis is performed using an inorganic hydroxide base.

10. The process of any one of claims 1 to 9, wherein said ester hydrolysis is performed using an inorganic hydroxide base selected from lithium hydroxide, sodium hydroxide, and potassium hydroxide.

11. The process of any one of claims 1 to 10, wherein said ester hydrolysis is performed using lithium hydroxide.

12. The process of any one of claims 1 to 11, wherein said ester hydrolysis is performed under aqueous conditions.

13. The process of any one of claims 1 to 12, wherein said ester hydrolysis is performed using lithium hydroxide, water, and ethanol.

14. A process for preparing a compound having the Formula II: 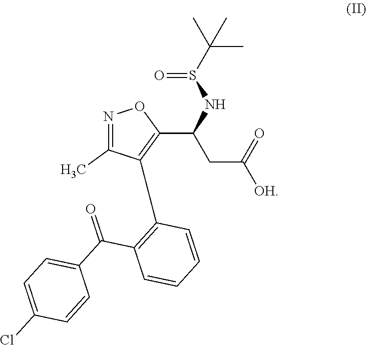 [SMILES: Cc1noc([C@H](CC(=O)O)N[S@@](=O)C(C)(C)C)c1-c1ccccc1C(=O)c1ccc(Cl)cc1]
said method comprising reacting a compound having the Formula I: 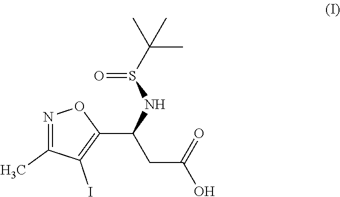 [SMILES: Cc1noc([C@H](CC(=O)O)N[S@@](=O)C(C)(C)C)c1I]
with a compound having the Formula B: 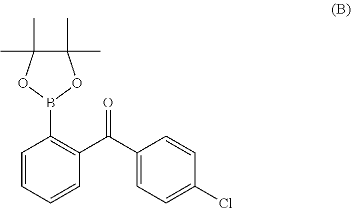 [SMILES: CC1(C)OB(c2ccccc2C(=O)c2ccc(Cl)cc2)OC1(C)C]
in the presence of a palladium source and a base.

15. The process of claim 14, wherein the palladium source is selected from Pd(Ph₃P)₄, Pd(OAc)₂, Pd₂(dba)₃, Pd(Ph₃)₂Cl₂, Pd(dppf)Cl₂.

16. The process of claim 14 or 15, wherein the palladium source is Pd(dppf)Cl₂.

17. The process of any one of claims 14 to 16, wherein the base is an inorganic base.

18. The process of any one of claims 14 to 17, wherein the base is selected from Na₂CO₃, K₂CO₃, Cs₂CO₃, t-BuOK, NaOEt, and K₃PO₄.

19. The process of any one of claims 14 to 18, wherein the base is K₃PO₄.

20. The process of any one of claims 14 to 19, wherein the reaction of the compound having the Formula I with the compound having the Formula B further comprises the addition of at least one organic solvent.

21. The process of any one of claims 14 to 20, wherein the reaction of the compound having the Formula I with the compound having the Formula B further comprises the addition of at least one aprotic organic solvent.

22. The process of any one of claims 14 to 21, wherein the reaction of the compound having the Formula I with the compound having the Formula B further comprises the addition of THF, 2MeTHF, MTBE, 1,4-dioxane, or DME.

23. The process of any one of claims 14 to 22, wherein the reaction of the compound having the Formula I with the compound having the Formula B further comprises the addition of 2MeTHF.

24. The process of any one of claims 14 to 23, further comprising reacting the compound having the Formula II with acid and reacting the resulting intermediate with base to form a compound having the Formula III:

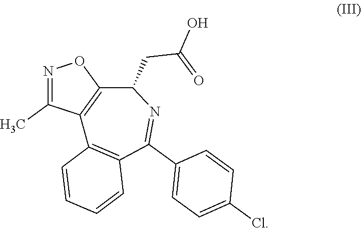 [SMILES: Cc1noc2c1-c1ccccc1C(c1ccc(Cl)cc1)=N[C@H]2CC(=O)O]

25. The process of claim 24, wherein the acid is an aqueous acid.

26. The process of claim 24 or 25, wherein the acid is selected from HCl, HBr, HI, H₃PO₄, and H₂SO₄.

27. The process of any one of claims 24 to 26, wherein the acid is HCl.

28. The process of any one of claims 24 to 27, wherein the base is an aqueous base.

29. The process of any one of claims 24 to 28, wherein the base is selected from Na₂CO₃, K₂CO₃, and K₃PO₄.

30. The process of any one of claims 24 to 29, wherein the base is K₃PO₄.

31. The process of any one of claims 14 to 30, wherein the compound having the Formula I is made via the process of any one of claims 1 to 13.

32. The compound 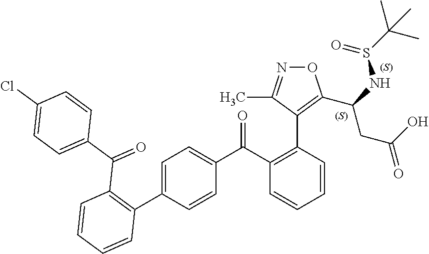
or a salt thereof.

33. A compound selected from: 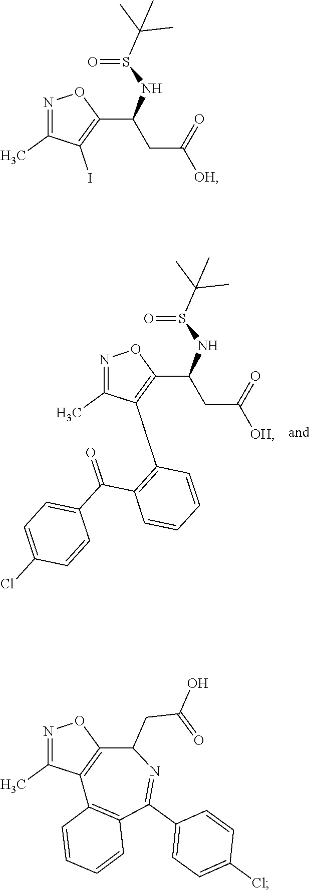 [SMILES: Cc1noc([C@H](CC(=O)O)N[S@@](=O)C(C)(C)C)c1I ; Cc1noc([C@H](CC(=O)O)N[S@@](=O)C(C)(C)C)c1Cc1ccccc1C(=O)c1ccc(Cl)cc1 ; Cc1noc2c1-c1ccccc1C(c1ccc(Cl)cc1)=NC2CC(=O)O]
or a salt of any of the foregoing.

34. The compound of claim 33, wherein the compound is 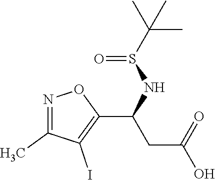 [SMILES: Cc1noc([C@H](CC(=O)O)N[S@@](=O)C(C)(C)C)c1I]
or a salt thereof.

35. The compound of claim 33, wherein the compound is 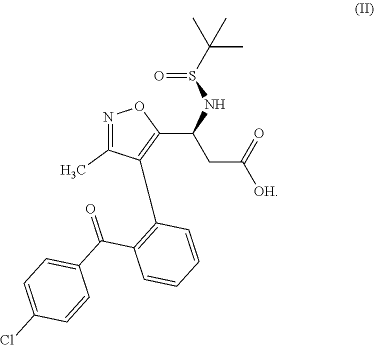 [SMILES: Cc1noc([C@H](CC(=O)O)N[S@@](=O)C(C)(C)C)c1-c1ccccc1C(=O)c1ccc(Cl)cc1]
or a salt thereof.

36. The compound of claim 33, wherein the compound is 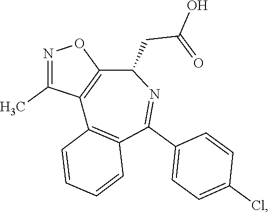 [SMILES: Cc1noc2c1-c1ccccc1C(c1ccc(Cl)cc1)=N[C@H]2CC(=O)O]
or a salt thereof.
